(19) United States
(12) Patent Application Publication
(10) Pub. No.: US 2008/0033530 A1
(43) Pub. Date: Feb. 7, 2008

(54) MARKER ALLOY

(75) Inventors: Bruno Zberg, Altdorf (CH); Joerg Loeffler, Zurich (CH); Bodo Gerold, Zellingen (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(73) Assignee: BIOTRONIK VI PATENT AG, Baar (CH)

(21) Appl. No.: 11/834,713

(22) Filed: Aug. 7, 2007

(30) Foreign Application Priority Data
Aug. 7, 2006 (DE) 10 2006 038 237.4

(51) Int. Cl. A61F 2/06; B05D 7/00; C22C 23/00; C22C 28/00

(52) U.S. Cl. 623/1.15; 420/402; 420/403; 420/407; 420/408; 420/411; 420/416; 427/2.24

(57) ABSTRACT

A marker alloy foreign implant made of a biodegradable metallic material and having the composition MgxYbyMz wherein x is equal to 10-60 atomic percent; y is equal to 40-90 atomic percent; z is equal to 0-10 atomic percent; M is one or more element selected from the group consisting of Ag, Zn, Au, Ga, Pd, Pt, Al, Sn, Ca, Nd, Ba, Si, and Ge; and wherein x, y, and z, together, and including contaminants caused by production, result in 100 atomic percent.

### PRIORITY CLAIMS

This patent application claims priority to German Patent Application No. 10 2006 038 237.4 filed Aug. 7, 2006, the disclosure of which is incorporated herein by reference in its entirety.

### FIELD

The present disclosure relates to a marker alloy for an implant made of a biodegradable metallic material and its use and a method for manufacturing the marker alloy.

### BACKGROUND

Implants have found uses in modern medical technology in manifold embodiments. They are used, for example, for supporting vessels, hollow organs, and duct systems (endovascular implants), for attaching and temporarily fixing tissue implants and tissue transplants, and for orthopedic purposes, for example, as nails, plates, or screws. Frequently, only a temporary support and/or retention function is necessary or desired until completion of the healing process or stabilization of the tissue. To avoid complications which result from the implants remaining permanently in the body, the implants must either be operatively removed or the implants must contain a material which is gradually degraded in the body, i.e., the material is biocorrodible. The number of biocorrodible materials based on polymers or alloys is growing continuously. Thus, inter alia, biocorrodible metal alloys of the elements magnesium, iron, and tungsten are known.

European Patent Application No. 1 270 023 describes a magnesium alloy which is suitable for endovascular and orthopedic implants. The alloy may contain up to 5 weight-percent rare earth elements. The biocorrodible metal alloys and polymers for medical implants known in the art have the disadvantage, however, that the biocorrodible metal alloys and polymers are not visible or are not visible to a satisfactory extent in the current x-ray methods. However, x-ray diagnosis is an important instrument precisely for postoperative monitoring of the healing progress or for checking minimally-invasive interventions. Thus, for example, stents have been placed in the coronary artery during acute myocardial infarction treatment for some years. Currently, a catheter which carries the stent in an unexpanded state is positioned in the area of the lesion of the coronary vascular wall. Subsequently, the stent either expands by self-expanding forces or by inflation of a balloon to prevent obstruction of the vascular wall in the expanded state. The procedure of positioning and expanding the stent must be continuously monitored by the cardiologist during the procedure.

X-rays in the energy range from 60 to 120 keV are typically employed in the medical field for use on the heart, typically, but not exclusively, in the range from 80 to 100 keV. Because the x-ray absorption coefficient is strongly dependent on the energy, the operating range is to be considered when selecting suitable marker materials. The absorption (intensity attenuation) of the x-rays may be described in simplified form using an exponential attenuation law.

$$\frac{I}{{I}_{0}} = exp [ - ( \frac{μ}{ρ} ) x ]$$

In the equation above, I is the measured intensity after the sample passage, I₀ is the intensity of the radiation before the sample passage, μ/ρ is the mass absorption coefficient, and x is the mass thickness of the sample. x may be calculated as the thickness t times the density of the material ρ,x=ρ*t. For alloys, the mass absorption coefficient is calculated by adding the components.

In the event of low absorption of the selected material in a given energy range of the x-ray absorption, improvement of the x-ray visibility may thus be achieved by increasing the material thickness; however, this measure rapidly reaches its limits, in particular, when marking filigree structures, as exists in stents.

Therefore, equipping implants with a marker in the form of a coating, a strip, an inlay, or a different type of design to improve the x-ray visibility is known. For example, metal strips made of gold or other noble metals are attached in specific areas of a stent.

German Patent Application No. 103 61 942 A1 describes a radiopaque marker for medical implants, which contains 10 to 90 weight-percent of a biocorrodible base component, in particular, from the group of elements consisting of magnesium, iron, and zinc. Furthermore, the marker contains 10 to 90 weight-percent of one or more radiopaque elements from the group consisting of I, Au, Ta, Y, Nb, Mo, Ru, Rh, Ba, La, Ce, Pr, Nd, Sm, Eu, Gd, Th, Dy, Ho, Er, Tm, Yb, Lu, Hf, Ta, W, Re, Os, and Bi as a marker component. The markers described are suitable in principle for use in biocorrodible implants, in particular, those made of biocorrodible magnesium alloys.

In implants made of biocorrodible metallic materials based on magnesium, iron, or tungsten, there are usually further requirements for the marker material:
the marker is not to be separated early from the main body of the implant by the corrosive processes, to avoid fragmentation and thus the danger of embolization;
the marker is not to degrade more rapidly than the main body, in order to still remain visible in later examination; however, at least partial in vivo degradation is to be provided;
the marker is to have sufficient x-ray density even at low material thicknesses; and
the marker material is to have no or only slight influence on the degradation of the main body.

However, when markers made of metallic materials are used on biocorrodible metallic main bodies, the special problem arises that, because of electrochemical interactions between the two materials, the degradation of the main body changes in a contact area between marker and main body, is typically accelerated. Furthermore, processing of the marker material is made more difficult because of the melting point of the base material, which is frequently low; processing methods such as soldering or laser welding, or also the immersion of the implant in a melt made of the marker material, are typically not possible.

### SUMMARY

The present disclosure provides several exemplary embodiments of the present invention.

One aspect of the present disclosure provides a marker alloy for an implant made of a biodegradable metallic material, comprising an alloy of the composition MgxYbyMz wherein x is equal to 10-60 atomic percent; y is equal to 40-90 atomic percent; z is equal to 0-10 atomic percent; M is one or more element selected from the group consisting of Ag, Zn, Au, Ga, Pd, Pt, Al, Sn, Ca, Nd, Ba, Si, and Ge; and x, y, and z, together, and including contaminants caused by production, result in 100 atomic percent.

Another aspect of the present disclosure provides a method for producing an x-ray marker for an implant made of a biodegradable magnesium alloy, the method comprising (a) providing a marker alloy having the formula MgxYbyMz wherein x is equal to 10-60 atomic percent; y is equal to 40-90 atomic percent; z is equal to 0-10 atomic percent; M is one or more element selected from the group consisting of Ag, Zn, Au, Ga, Pd, Pt, Al, Sn, Ca, Nd, Ba, Si, and Ge; and x, y, and z, together, and including contaminants caused by production, result in 100 atomic percent; and (b) forming an x-ray marker for an implant made of a biodegradable magnesium alloy incorporating the marker alloy of step (a).

A further aspect of the present disclosure provides an implant incorporating a marker alloy, comprising a composition having the formula MgxYbyMz wherein x is equal to 10-60 atomic percent; y is equal to 40-90 atomic percent; z is equal to 0-10 atomic percent; M is one or more element selected from the group consisting of Ag, Zn, Au, Ga, Pd, Pt, Al, Sn, Ca, Nd, Ba, Si, and Ge; and x, y, and z, together, and including contaminants caused by production, result in 100 atomic percent.

### DETAILED DESCRIPTION

According to one exemplary embodiment, the marker alloy is distinguished by (i) its low melting point (approximately 450° C. to 800° C. for the specified alloy compositions) and special suitability for typical thermal processing methods, such as soldering or laser welding, (ii) a homogeneous microstructure without intermetallic phases, which simplifies processability, and (iii) (at least partial) biocorrodibility. Both a homogeneous structure (mixed crystal) and the occurrence of intermetallic phases may be controlled by suitable selection of the production parameters. The production parameters essentially include, but are not limited to, the composition of the melt, the temperature of the marker melt and of the substrate, the surrounding atmosphere (inert, e.g., vacuum or argon gas; reactive, e.g., nitrogen) and pressure, and the cooling rate and further following heat treatment measures, which are, in turn, essentially characterized by the temperature and heating and cooling rates and the surrounding atmosphere.

Preferably, x equals 25 to 40 atomic percent and y equals 60 to 75 atomic percent, and especially preferably x equals 28 to 35 atomic percent and y equals 65 to 72 atomic percent. Particularly preferably, the marker alloy corresponds to the composition Mg31.5Yb68.5. It has been shown that marker alloys of the cited compositions have a sufficiently high mean mass absorption coefficient for the medical technology x-ray energy range of 80 keV to 100 keV and a melting temperature which is below the melting point of the biocorrodible magnesium alloys used up to this point for the main body of the implant. Furthermore, marker alloys of the cited composition are also stable for a sufficiently long time in aqueous or physiological solution for the intended purposes.

The addition of the component M is optional and is particularly used for lowering the melting point of the outlet. Preferably z equals 3 to 8 atomic percent.

The alloy composition Mg31.5Yb68.5 is a eutectic mixture, whose melting point is approximately 496° C., while, for example, the biocorrodible magnesium alloy WE43 has a melting point of approximately 590° C. A required material thickness of 51 μm for an attenuation of the intensity to the factor 0.86 may be calculated from the density of this marker alloy (5.9 g/cm3) and its mean mass absorption coefficient in the energy range from 80 to 100 keV (5.98 cm2/g). This value is significantly less than the wall thickness of typical magnesium stents. The cited factor corresponds to an attenuation coefficient as is observed in gold-coated steel stents, a thickness of the steel being 70 μm and a thickness of the gold coating being 14 μm. In other words, a material thickness of the marker alloy was calculated which is necessary to obtain the same intensity attenuation as in the steel/gold composite and the ascertained value of 51 μm illustrates that this marker alloy is suitable for the filigree structures of stents.

A special feature of the marker alloy is that the electronegativity of ytterbium is less than that of magnesium, so that an acceleration of the corrosion of the main body in the contact area to the marker material by the formation of local elements is prevented.

The biocorrodible metallic material is preferably, but not exclusively, a biocorrodible alloy selected from the group of elements consisting of magnesium, iron, and tungsten, in particular, a biocorrodible magnesium alloy, such as WE43. The cited elements are provided in the alloy as the main component, i.e., the mass proportion is greatest in comparison to the other elements present in the alloy. The mass proportion of the cited elements in the biocorrodible alloys is preferably more than 50 weight-percent, in particular, more than 70 weight-percent.

A biocorrodible magnesium alloy of the composition rare earth metals 5.2-9.9 weight-percent, yttrium 3.7-5.5 weight-percent, and the remainder less than 1 weight-percent, magnesium making up the proportion of the alloy to 100 weight-percent, is especially suitable as the implant material. This magnesium alloy has already confirmed its special suitability experimentally and in initial clinical trials, i.e., the magnesium alloy displays a high biocompatibility, favorable processing properties, good mechanical characteristics, and corrosion behavior adequate for the intended uses. For purposes of the present disclosure, the collective term “rare earth metals” includes scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70), and lutetium (71).

The biocorrodible alloys of the elements magnesium, iron, or tungsten are to be selected in composition in such a way that the elements are biocorrodible. For purposes of the present disclosure, alloys are referred to as biocorrodible when degradation occurs in a physiological environment, which finally results in the entire implant or the part of the implant made of the material losing its mechanical integrity. Artificial plasma, as has been previously described according to EN ISO 10993-15:2000 for biocorrosion assays (composition NaCl 6.8 g/l, CaCl2 0.2 g/l, KCl 0.4 g/l, MgSO4 0.1 g/l, NaHCO3 2.2 g/l, Na2HPO4 0.126 g/l, NaH2PO4 0.026 g/l), is used as a testing medium for testing the corrosion behavior of an alloy under consideration. For this purpose, a sample of the alloy to be assayed is stored in a closed sample container with a defined quantity of the testing medium at 37° C. At time intervals, tailored to the corrosion behavior to be expected, of a few hours up to multiple months, the sample is removed and examined for corrosion traces by techniques known to those skilled in the art. The artificial plasma according to EN ISO 10993-15:2000 corresponds to a medium similar to blood and represents a possibility for reproducibly simulating a physiological environment.

The x-ray marker is preferably provided in solid embodiment as a solid material. Alternatively, the x-ray marker may also be embedded as a powder in an inorganic carrier matrix.

The implant is preferably a stent, in particular, made of a magnesium or iron alloy (e.g., the magnesium alloy WE43). There is a significant need for marker materials, which result from the special requirements for the design and material of the stent.

In an exemplary embodiment, the implant is produced from the marker material.

### EXAMPLES

### Example 1

An alloy was produced by joint melting the alloy components in a graphite or boron nitride crucible, concretely by joint melting of 31.5 atomic percent magnesium and 68.5 atomic present ytterbium. Because both magnesium and ytterbium have a very high tendency to oxidize and low vaporization enthalpies, the melting process was performed under protective gas and with slight overpressure.

### Example 2

A stent made of the magnesium alloy WE43 (containing 93 weight-percent magnesium, 4 weight-percent yttrium (W) and 3 weight-percent rare earth metals besides yttrium (E)) was immersed on both sides at the ends up to a depth of approximately 1 mm and for 1-2 seconds in a melt made of Mg31.5Yb68.5 and was subsequently cooled. The cooled layer made of the marker material was approximately 50 μm thick.

All patents, patent applications and publications referred to herein are incorporated by reference in their entirety.

What is claimed is:

1. A marker alloy for an implant made of a biodegradable metallic material, comprising:
an alloy of the composition MgxYbyMz
wherein
x is equal to 10-60 atomic percent;
y is equal to 40-90 atomic percent;
z is equal to 0-10 atomic percent;
M is one or more element selected from the group consisting of Ag, Zn, Au, Ga, Pd, Pt, Al, Sn, Ca, Nd, Ba, Si, and Ge; and
x, y, and z, together, and including contaminants caused by production, result in 100 atomic percent.

2. The marker alloy of claim 1, wherein
x equals 25-40 atomic percent;
y equals 60-75 atomic percent; and
z equals 0-10 atomic percent.

3. The marker alloy of claim 2, wherein
x equals 28-35 atomic percent;
y equals 65-72 atomic percent; and
z equals 0-10 atomic percent.

4. The marker alloy of claim 3, wherein the alloy comprises Mg31.5Yb68.5.

5. The marker alloy of claim 1, wherein the biodegradable metallic material of the implant is an alloy of an element selected from the group consisting of magnesium, iron, and tungsten.

6. A method for producing an x-ray marker for an implant made of a biodegradable magnesium alloy, the method comprising:
(a) providing a marker alloy having the formula MgxYbyMz wherein
x is equal to 10-60 atomic percent;
y is equal to 40-90 atomic percent;
z is equal to 0-10 atomic percent;
M is one or more element selected from the group consisting of Ag, Zn, Au, Ga, Pd, Pt, Al, Sn, Ca, Nd, Ba, Si, and Ge; and
x, y, and z, together, and including contaminants caused by production, result in 100 atomic percent; and
(b) forming an x-ray marker for an implant made of a biodegradable magnesium alloy incorporating the marker alloy of step (a).

7. The marker alloy of claim 1, wherein the implant is a stent.

8. An implant incorporating a marker alloy, comprising:
a composition having the formula MgxYbyMz
wherein
x is equal to 10-60 atomic percent;
y is equal to 40-90 atomic percent;
z is equal to 0-10 atomic percent;
M is one or more element selected from the group consisting of Ag, Zn, Au, Ga, Pd, Pt, Al, Sn, Ca, Nd, Ba, Si, and Ge; and
x, y, and z, together, and including contaminants caused by production, result in 100 atomic percent.

9. The implant of claim 8, wherein
x equals 25-40 atomic percent;
y equals 60-75 atomic percent; and
z equals 0-10 atomic percent.

10. The implant of claim 9, wherein
x equals 28-35 atomic percent;
y equals 65-72 atomic percent; and
z equals 0-10 atomic percent.

11. The implant of claim 10, having the composition Mg31.5Yb68.5.

12. The implant of claim 8, wherein the biodegradable metallic material of the implant is an alloy of an element selected from the group consisting of magnesium, iron, and tungsten.
